(19) United States
(12) Patent Application Publication
(10) Pub. No.: US 2026/0283698 A1
(43) Pub. Date: Sep. 24, 2026

(54) BINARY ENCODING FOR WIRE IDENTIFICATION

(71) Applicant: ASAHI INTECC CO., LTD., Seto-shi (JP)

(72) Inventor: Hirobumi WATANABE, Irvine, CA (US)

(21) Appl. No.: 19/459,511

(22) Filed: Jan. 26, 2026

Related U.S. Application Data
(60) Provisional application No. 63/774,965, filed on Mar. 20, 2025.

(51) Int. Cl. A61B 34/20

(52) U.S. Cl. CPC A61B 34/20; A61B 2034/2051; A61B 2034/2061

(57) ABSTRACT

Embodiments of the present disclosure relate to a sensor guidewire and methods for fabricating the same. An example sensor guide wire includes a core wire extending between a first end and a second end; and a plurality of wires in a helical arrangement around the core wire, wherein: a first subset of the wires comprises an outer surface comprising a first identification feature; a second subset of the wires comprises an outer surface comprising a second identification feature that is different from the first identification feature; respective wires of the first and second subsets are positioned in accordance with at least one binary permutation group of the first identification feature or the first identification feature and the second identification feature to differentiate the wires from one another; and a quantity of the at least one binary permutation group is based on a total quantity of the wires.

### CROSS-REFERENCE TO RELATED APPLICATIONS

This application claims the benefit of and priority to U.S. Application No. 63/774,965, filed Mar. 20, 2025, the disclosure of which is incorporated herein by reference in its entirety.

### TECHNICAL FIELD

This application generally relates to sensor guide wires and methods for fabricating the same.

### BACKGROUND

Guidewires for medical use may have a number of sensors or sensor assemblies integrated directly into the guidewire. Such sensor-equipped guidewires may be adapted for measuring various physiological parameters within a patient's body. As the diameter of the guidewire decreases, accommodation of certain types of sensors or incorporation of plural sensors may be limited due to a relatively small space provided by the sensor guide wire. A guidewire may include multiple conductive wires connected to elements spaced along the guidewire; however, differentiation and, therefore, management, between wires may become difficult.

### BRIEF SUMMARY

Embodiments of the present disclosure relate to sensor guide wires and methods for fabricating the same. An example sensor guide wire of the present disclosure may include a core wire extending between a first end and a second end, the first end and the second end defining a length of the sensor guide wire; and a plurality of wires in a helical arrangement around the core wire, wherein: a first subset of the plurality of wires comprises an outer surface comprising a first identification feature; a second subset of the plurality of wires comprises an outer surface comprising a second identification feature that is different from the first identification feature; respective wires of the first subset and respective wires of the second subset are positioned in the helical arrangement in accordance with at least one binary permutation group of the first identification feature or the first identification feature and the second identification feature to differentiate individual ones of the plurality of wires from one another; and a quantity of the at least one binary permutation group is based on a total quantity of the plurality of wires.

### BRIEF DESCRIPTION OF THE FIGURES

Having thus described the embodiments of the disclosure in general terms, reference now will be made to the accompanying drawings, which are not necessarily drawn to scale, and wherein:

FIG. 1A shows a partial perspective view of an example sensor guide wire in accordance with some embodiments of the present disclosure;

FIG. 1B shows a cross section of an example sensor guide wire in accordance with some embodiments of the present disclosure;

FIG. 1C shows an example sensor guide wire's helical arrangement of wires;

FIG. 1D shows an example sensor guide wire observed with an exemplary hypotube;

FIG. 2 shows a perspective view of an example sensor guide wire in accordance with some embodiments of the present disclosure;

FIGS. 3 and 4 show diagrams of an example technique for configuring wires of a sensor guide wire for individual identification in accordance with some embodiments of the present disclosure; and

FIG. 5 shows a flowchart of an example process for fabricating and using a sensor guide wire in accordance with some embodiments of the present disclosure.

### DETAILED DESCRIPTION

Some embodiments of the present invention will now be described more fully hereinafter with reference to the accompanying drawings, in which some, but not all, embodiments of the invention are shown. Like reference numerals refer to like elements throughout. Indeed, various embodiments of the invention may be embodied in many different forms and should not be construed as limited to the embodiments set forth herein. Rather, these embodiments are provided so that this disclosure will satisfy applicable legal requirements.

As used herein, the term “or” is used in both the alternative and conjunctive sense, unless otherwise indicated. The term “along,” and similarly utilized terms, means near or on, but not necessarily requiring directly on an edge or other referenced location. The terms “approximately” refer to within manufacturing and/or engineering design tolerances for the corresponding materials and/or elements unless otherwise indicated. Thus, use of any such aforementioned terms, or similarly interchangeable terms, should not be taken to limit the spirit and scope of embodiments of the present invention.

As used herein, the term “distal” shall refer to a direction extending toward a target site of a subject (e.g., away from a user of the sensor guide wire). As used herein, the term “proximal” shall refer to a direction extending away from a target site of a subject (e.g., toward a user of the sensor guide wire). For example, a distal end of a sensor guide wire may refer to an end of the sensor guide wire that is directed towards or positioned at a target site within a subject, and a proximal end of the sensor guide may refer to an opposite end of the sensor guide wire that is manipulated by a user (e.g., potentially extending outside of the target site or subject entirely).

The terms “about” or “approximately” as used herein when referring to a measurable value—such as, for example, length, width, height, separation and the like—are meant to encompass variations of ±20%, ±10%, ±5%, ±1%, ±0.5%, or even ±0.1% of the specified amount. A range provided herein for a measurable value may include any other range and/or individual value therein.

### Example Sensor Guide Wire

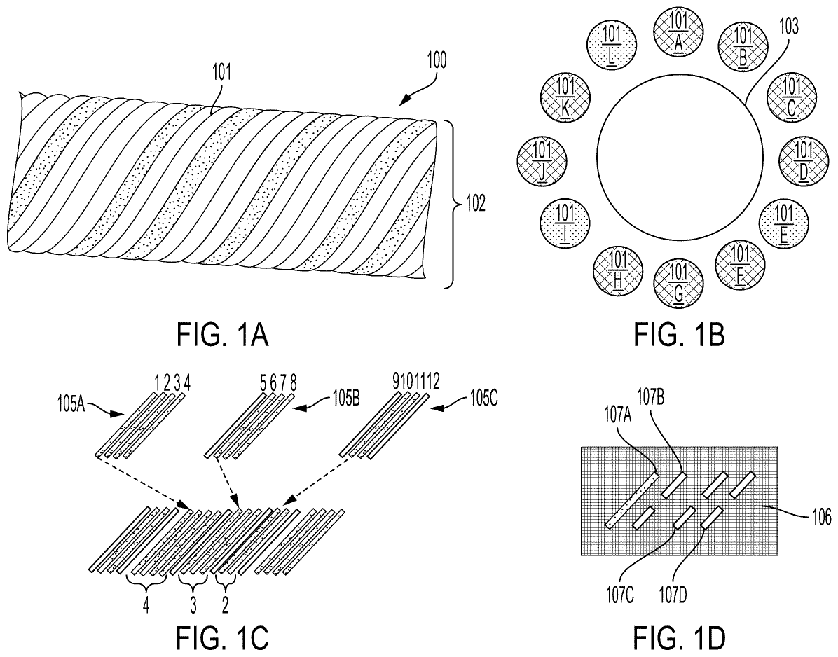

With reference to FIG. 1A, a partial perspective view of an example sensor guide wire 100 is illustrated. In some embodiments, the sensor guide wire 100 includes a plurality of wires 101 in a helical arrangement 102. In various embodiments, the helical arrangement 102 provides an optimal balance of torque force, torque response, durability, and rigidity. For example, as compared to existing guide wire approaches, the helical arrangement 102 may demonstrate greater ability to transmit torque along its length (e.g., from a proximal end away from the subject to a distal end toward the subject). In various embodiments, the sensor guide wire 100 includes a core wire 103 within the helical arrangement 102. For example, as shown in FIG. 1B, the helical arrangement 102 may comprise a central void within which the core wire 103 is located. In some embodiments, the plurality of wires 101 include 4 wires, 8 wires, 12 wires, 16, wires, 24 wires, and/or the like, with greater or lesser numbers of wires being contemplated and within the scope of the disclosure. Further example embodiments of the helical arrangement 102, wires 101, and core wire 103 are shown in U.S. Application No. 63/709,580, filed Oct. 21, 2024, entitled “SENSOR GUIDE WIRE,” the disclosure of which is incorporated herein by reference in its entirety.

In various embodiments, respective wires 101 comprise stainless steel material. Additionally, or alternatively, in some embodiments, a wire 101 comprises enamel-coated copper material. In some embodiments, a diameter of the wire 101 is approximately 0.24 to 0.40 mm. For example, a diameter of the wire 101 may be 0.254 mm. As another example, a diameter of the wire 101 may be 0.355 mm. In some embodiments, a cross-sectional area of the wire 101 is approximately 0.045 to 0.126 square mm. For example, a cross-sectional area of the wire 101 may be 0.05 square mm. As another example, a cross-sectional area of the wire 101 may be 0.099 square mm. In some embodiments, a wire 101 comprises a layer of a platinum-iridium (Pt/Ir) alloy. In various embodiments, the Pt/Ir layer is configured to conduct electrophysiological signals measured by electrodes of the sensor guide wire 100. The Pt/Ir layer may improve the ability of the wire 101 to transmit torque along its length, thereby increasing the overall torque transmission capability of the helical arrangement 102. In some embodiments, a thickness of the Pt/Ir layer is approximately 10.0 to 50.0 microns. For example, a thickness of the Pt/Ir layer may be 25.0 microns. Additionally, the Pt/Ir layer may render the wire 101 radiopaque such that the wire 101 may be visualized via a radiation-based imaging technique, such as X-ray.

In some embodiments, a wire 101 comprises a layer of polytetrafluoroethylene (PTFE). For example, the plurality of wires 101 may be individually coated with a film of PTFE. In some embodiments, the PTFE layer includes a thickness of approximately 20.0 to 60.0 microns. For example, the PTFE layer may include a thickness of 33.0 microns. As another example, the PTFE layer may include a thickness of 4.0 microns. The Pt/Ir layer be covered by the PTFE layer. In this manner, the PTFE layer may electrically insulate the wires 101 from one another, the core wire 201, and the external environment. In some embodiments, the insulation of the wires 101 reduces resistance of the wire as compared to existing approaches, which may result in improved impedance properties. Additionally, the insulation may reduce electrical noise and interference. In some embodiments, the materials of the wire 101 demonstrate respective longitudinal resistivities of approximately 70 microohm-centimeters (stainless steel), 1.7 microohm-centimeters (copper), 10.6 microohm-centimeters (platinum), and 4.71 microohm-centimeters (iridium).

In some embodiments, a respective wire 101 includes a cross section including an ovoid or circular shape. In some embodiments, a diameter of the wire 101, including a Pt/Ir layer, is approximately 0.15 to 0.40 mm. For example, a diameter of the wire 101, including a Pt/Ir layer is 0.25 mm. In some embodiments, the diameter of the wire 101, further including an outer PTFE layer, is approximately 0.2 to 0.5 mm. For example, the diameter of the wire 101, further including an outer PTFE layer, may be 0.33 mm. In some embodiments, at a distal end of a distal portion of the sensor guide wire 100, the helical arrangement 102 includes an outer diameter of approximately 0.2 to 0.4 mm and an inner diameter of approximately 0.1 to 0.3 mm. For example, at distal end of the distal portion of the sensor guide wire 100, the helical arrangement 102 may include an outer diameter of 0.250 mm and an inner diameter of 0.186 mm.

In some embodiments, the core wire 103 and helical arrangement 102 taper in diameter from a distal portion of the sensor guide wire 100 (away from a target site) toward a proximal portion of the sensor guide wire (toward a target site). For example, respective diameters of the core wire 103 and helical arrangement 102 may taper toward a proximal tip. In some embodiments, the core wire 103 includes stainless steel material. Additionally, or alternatively, in some embodiments, the core wire 103 includes one or more nickel-titanium alloys. For example, the core wire 103 may include nitinol, and/or the like.

In various embodiments, a respective wire 101 includes a first end and a second end opposite the first end. In some embodiments, the first end comprises a sensor configured to receive and conduct an electrophysiological signal along a length of the wire to the second end. In some embodiments, the second end is configured to conduct the electrophysiological signal to a measurement device. Thus, in various embodiments, a respective wire 101 defines or comprises a sensing element that may extend into a target region within a body of a subject and project out of the subject such that the electrophysiological signal may be conducted from the wire to a measurement device. In some embodiments, all or a subset of the wires 101 comprising the helical arrangement 102 define or comprise a sensor arrangement. For example, as shown in FIG. 1B, the sensor guide wire 100 may include twelve wires 101A-L in a helical arrangement 102 around a core wire 103. Each or a subset of the wires 101A-L may define an independent electrode configured to conduct one or more electrophysiological signals along a length of the wire from the first end to the second end.

As the number of wires 101 in the helical arrangement 102 increases, the difficulty of differentiating between the sensor embodied by each wire 101 increases. One approach to enabling differentiation of the wires includes coating each wire in a different color. However, such a technique may undesirably increase a cost of manufacturing the sensor guide wire 100. Additionally, the differentiability of colors may be limited as the quantity of wires 101 further increases. Another approach may include identifying wires based on distance measurements and a jig. However, such techniques may demonstrate poor precision and accuracy in practice due to the micro-dimensionality of the wires and micromovements of the subject while the sensor guide wire 100 is inserted within the body. Another approach may include identifying a target wire via thermal imaging, such as by passing current through a wire and observing the wire under infrared imaging. However, the approach may demonstrate limitations in vivo due to surrounding body heat and interference and safety limitations of applying an external current through the sensor guide wire 100.

In various embodiments, the present sensor guide wire overcomes these challenges, and others, to enable differentiability of wires 101 in the helical arrangement 102. In some embodiments, the present techniques introduce and utilize a first identification feature, and a second identification feature applied to a first subset of the wires 101 and a second subset of the wires 101, respectively. Further, the present techniques may arrange wires 101 relative to one another in the helical arrangement in accordance with one or more binary permutation groups, enabling identification of individual wires 101 based on decoding of the binary groups. In this manner, the techniques may enable the respective electrodes (or other sensing elements) defined by the wires 101 to be precisely and accurately identified while minimizing resource costs of the sensor guide wire and technical complexity of the means for individually differentiating between the wires 101.

As shown in FIG. 1B, a first subset of wires 101 (e.g., 101A, 101B, 101C, 101D, 101F, 101G, 101H, 101J, 101K) may include an outer coating comprising a first identification feature (depicted as a cross-hatching pattern for exemplary purposes). A second subset of wires 101 (e.g., 101E, 101I, 101L) may include an outer coating comprising a second identification feature (depicted as a stippling pattern for exemplary purposes). In some embodiments, the first identification feature comprises a first color coating (e.g., red) and the second identification feature comprises a second color coating that is different from the first color (e.g., black). Alternatively, or additionally, in some embodiments, the first identification feature comprises a first surface texture (e.g., bumps) and the second identification feature comprises a second surface texture that is different from the first texture (e.g., smooth, ridged, waved, and/or the like). Thus, in various embodiments, the plurality of wires 101 include a first subset of wires 101 and a second subset of wires 101 that are visually differentiable from the first subset of wires 101.

In various embodiments, within the helical arrangement 102, the wires 101 are positioned relative to one another in accordance with one or more binomial permutation groups to enable differentiability and individual identification of each wire. For example, FIG. 1B depicts an example embodiment in which the sensor guide wire 100 comprises twelve wires 101A-L, where wires 101E, 101I, 101L comprise a first identification feature (e.g., black outer coating) and wires 101A, 101B, 101C, 101D, 101F, 101G, 101H, 101J, 101K comprise a second identification feature (e.g., red outer coating). By arranging the black wires and red wires in accordance with binomial permutation groups based on the total quantity of wires, each wire 101A-L may be individually differentiable from other wires in the helical arrangement 102 based on the encoding schema provided by the binomial permutation groups. In FIG. 1C, the helical arrangement 102 comprises a first, a second, and a third binomial permutation group 105A, 105B, 105C. The first binomial permutation group 105A may include a subset of wires comprising identical identification features, such as four wires from the second subset and no wires from the first subset. For example, the first binomial permutation group 105A may include wires 101A, 101B, 101C, 101D. In this manner the, wires 101A-D may be differentiated in the helical arrangement 102 by observing a sequence of four consecutive wires that comprise the second identification feature (e.g., red outer coating). Further, the wires 101A-D may be individually identified based on their respective position in the consecutive sequence.

The second binomial permutation group 105B may include a single wire from first subset (e.g., comprising black outer coating) and three wires from the second subset (e.g., comprising red outer coating). In the second binomial permutation group 105B, the wires from the second subset may be positioned adjacent one another. For example, the second binomial permutation group 105B may include a sequence of the wires 101E and wires 101F, 101G, 101H. In this manner, the second binomial permutation group 105B may be differentiated in the helical arrangement 102 by observing a sequence of three consecutive wires that comprise the second identification feature. Further, the wires 101E-H in the second binomial permutation group 105B may be individually identified by their respective positions relative to one another.

The third binomial permutation group 105C may include two wires from the first subset and two wires from the second subset. In the third binomial permutation group 105C, the two wires from the second subset may be arranged adjacent to one another. For example, the third binomial permutation group 105C may include a sequence comprising a first wire from the first subset, wire 101I, two consecutive wires from the second subset, wires 101J, 101K, and a second wire from the first subset, wire 101L. In this manner, the third binomial permutation group 105C may be differentiated in the helical arrangement 102 by observing a sequence of precisely two consecutive wires comprising the second identification feature. Further, the wires 101I-L may be individually identified based at least in part on their respective positions within the subset (e.g., by count of position or relative position, referring to the identification features of wires adjacent to the target wire).

In various embodiments, a hypotube 106 is used to evaluate the position of wires 101 within the helical arrangement 102, such as to differentiate binomial group permutations from one another and/or individually identify a wire 101. For example, as shown in FIG. 1D, the hypotube 106 may be slid over the sensor guide wire 100. The hypotube 106 may include a plurality of windows 107A-D spaced apart from one other such that the windows 107A-D may be aligned over a predetermined quantity of wires, with each wire occupying a respective window. In doing so, the hypotube 106 may facilitate observation and evaluation of the respective sequences of identification features comprised by the wires aligned to the windows 107A-D. In this manner, sequences indicative of particular binomial permutation groups may be recognized, and individual wires may be precisely and accurately identified. In some embodiments, one of the plurality of windows 107A-D is configured to enable ablation of a portion of a wire 101 that is aligned within the window (e.g., controlled removal of material, such as insulation). In this manner, the conductive inner portion of the wire 101 may be accessed to pass electrophysiological signals conducted by the wire 101 to a measurement device.

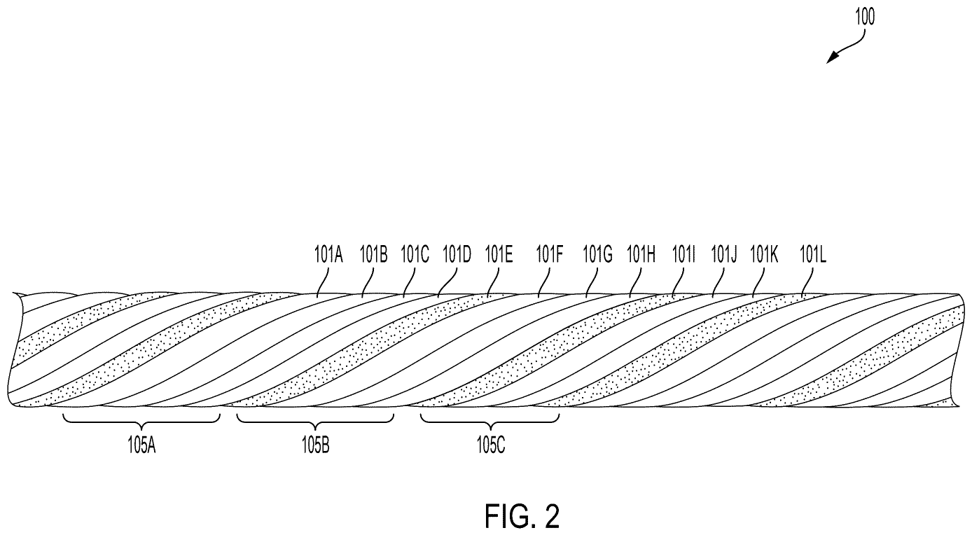

FIG. 2 shows a side view of an example sensor guide wire 100. In some embodiments, the helical arrangement includes wires 101A, 101B, 101C, 101D, 101E, 101F, 101G, 101H, 101I, 101J, 101K, 101L. In some embodiments, the wires 101A-L comprise or define twelve independent electrodes. For example, electrophysiological signals may be conducted along twelve independent channels (e.g., to a receiving computing device). It will be understood and appreciated that the number of wires and electrodes is exemplary and additional wires and electrodes defining additional electrophysiological sensors are contemplated. As further depicted, the wires 101A-L may be differentiated from one another based at least in part on partitioning into binomial permutation groups 105A, 105B, 105C. As used herein “binary” may refer to a respective wire 101 comprising one of two identification features. For example, a first identification feature (e.g., black outer coating) may represent a first binary value, such as 0. A second identification feature (e.g., red outer coating) may represent a second binary value, such as 1. In this manner, respective sequences of wires 101 may be provided in the helical arrangement 102 in accordance with different binary representations of natural numbers. Thus, the wires 101 may be partitioned into different binomial permutation groups representing different natural numbers, where a sum of the natural numbers represented is equal to a total quantity of the wires 101.

For example, as depicted in FIGS. 1 and 2, the sensor guide wire 100 may include twelve wires in a helical arrangement 102. The first binomial permutation group 105A may represent a first natural number (e.g., three), the second binomial permutation group 105B may represent a second natural number (e.g., four), and the third binomial permutation group 105C may represent a third natural number (e.g., five). A sum of the represented natural numbers (e.g., three, four, five) may be equal to the total quantity of wires (e.g., twelve).

In various embodiments, a binary permutation-based technique is provided for
for configuring the wires 101 of the helical arrangement to enable precise and accurate differentiation and identification of individual wires. In various embodiments, the technique decomposes the total quantity of wires into a sequence of natural numbers, which may be represented via binomial permutation groups formed from subsets of wires having a first identification feature or a second identification feature. In some embodiments, the technique includes determining a nearest sum (k) of consecutive natural numbers that is equal to or greater than the total quantity of wires. Table 1 shows example summations of consecutive natural numbers (n). For example, a sum of consecutive numbers one to four may be equal to ten. In various embodiments, the technique includes subtracting from the determined sequence of natural numbers the difference (a) between the nearest sum k and the total quantity of wires. The difference (a) may be removed from the determined sequence of consecutive natural numbers n, yielding a second sequence of natural numbers (which may or may not be consecutive) with a sum equal to the total quantity of wires. In various embodiments, the technique includes determining one or more binomial permutation groups for representing the second sequence of natural numbers.

TABLE 1

| n | Σnᵢ |
|---|---|
| 1 | 1 |
| 2 | 3 |
| 3 | 6 |
| 4 | 10 |
| 5 | 15 |
| 6 | 21 |
| 7 | 28 |
| 8 | 36 |
| 9 | 45 |
| 10 | 55 |
| 11 | 66 |
| 12 | 78 |
| 13 | 91 |
| 14 | 105 |
| 15 | 120 |
| 16 | 136 |
| 17 | 153 |
| 18 | 171 |
| 19 | 190 |
| 20 | 210 |
| 21 | 231 |

In other words, the binary permutation-based technique may be performed to decompose a large number (e.g., 12, 20, and/or the like) into smaller natural numbers. The technique may include using a summation of natural numbers as reference points in accordance with Table 1. In various embodiments, the technique may include determining, in the second column of Table 1, the number nearest and greater or equal to the target number (e.g., 21 in a scenario where the target number of wires is 20). The technique may include identifying, in the first column of Table 1, the natural number associated with the determined sum (e.g., 6 in a scenario of 21). The technique may include determining, based on the summation combination of the two determined numbers (e.g., 21=1+2+3+4+5+6) a summation of natural numbers that equals the target number (e.g., in a scenario of 20, removing the number 1 to obtain a summation of 2+3+4+5+6). The combination may be performed in accordance with Equation 1, where a represents the difference between the target number and the nearest greater number. For example, where the target number is 18, the nearest greater number may be 21, providing a difference of 3. The summation associated with 21 may be 1+2+3+4+5+6, therefore 3 may be removed from the summation to obtain a sequence of natural numbers equaling 18 (e.g., 1+2+4+5+6 or 3+4+5+6).

$${\sum }_{i = 1}^{n} k - a = {\sum }_{i = 1}^{n - 1} k + n - a \qquad ( Equation 1 )$$

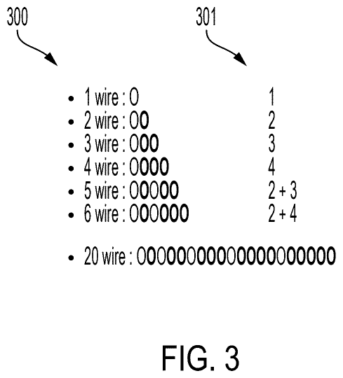

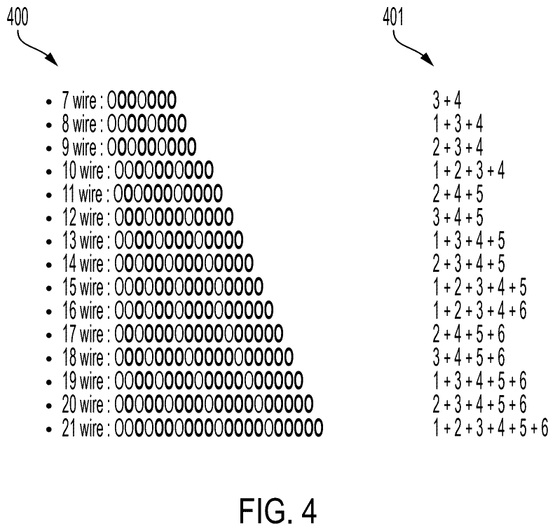

FIGS. 3 and 4 show example diagrams of a technique for determining binomial permutation groups to represent the second sequence of natural numbers. In various embodiments, a respective binomial permutation group comprises binomial values (e.g., zeros and ones) in the form of identification features (e.g., black or red, textured or smooth, and/or the like). In some embodiments, the technique includes determining one or more binomial permutation groups based at least in part on binomial representation schemas 300, 400 that defines binomial representation relationships between sequences of wires of the first identification feature and wires of the second identification feature and natural number sequences 301, 401, which equate to total numbers of wires 101 in the helical arrangement. For example, as shown in FIG. 4, a total quantity of wires 101 in a sensor guide wire may be twelve. The total quantity, twelve, may be represented by a sequence of natural numbers that, when summed, equal the quantity. For example, the twelve wires 101 may be represented by a string of natural numbers three, four, and five.

In accordance with the binomial representation schemas 300, 400, a plurality of binomial permutation groups may be defined, where each binomial permutation group represents one of the natural numbers in the determined sequence. For example, a first binomial permutation group may represent three, a second binomial permutation group may represent four, and a third binomial representation group may represent five. The number may be encoded by combinations of wires from the first subset of wires 101 having a first identification feature and the second subset of wires 101 having the second identification feature). For example, a binomial permutation group representing the number three may comprise one black wire and two adjacent red wires, a binomial permutation group representing the number four may comprise three adjacent red wires and a black wire, and a binomial permutation group representing the number three may comprise four consecutive red wires a black wire.

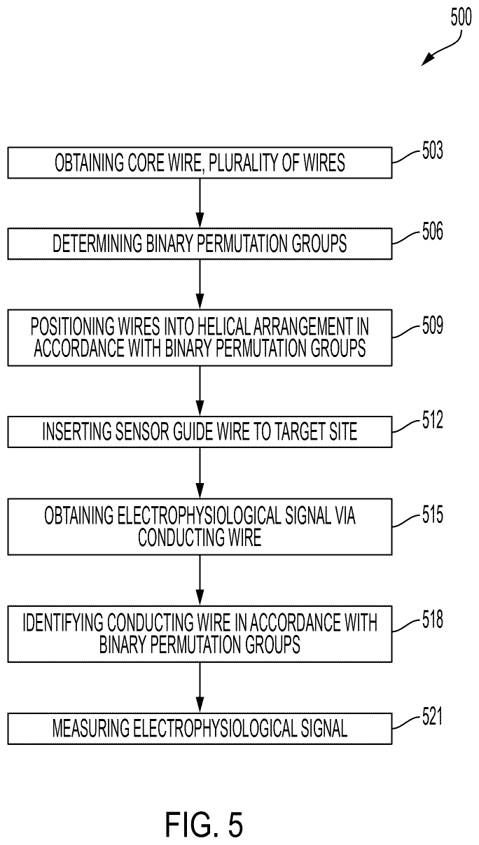

Referring now to FIG. 5, shown is an example process 500 for fabricating and using a sensor guide wire (e.g., a sensor guide wire 100, and/or the like) in accordance with some embodiments of the present disclosure. In various embodiments, the process 500, or a subset of operations/blocks thereof, is performed to fabricate a sensor guide wire 100 comprising a plurality of wires 101 in a helical arrangement 102 around a core wire 103. In some embodiments, the process 500, or a subset of operations/blocks thereof, is performed to measure one or more electrophysiological signals of a subject using the sensor guide wire 100.

At block 503, the process 500 includes obtaining a core wire and a plurality of wires that may be formed into a helical arrangement around the core wire. For example, a core wire 103 and a plurality of wires 101 may be obtained. In some embodiments, the wires 101 comprise one or more conductive materials. In some embodiments, the wires 101 and core wire 103 are individually insulated. In various embodiments, the wires 101 include a first subset comprising a first identification feature and a second subset comprising a second identification feature that is different from the first identification feature.

At block 506, the process 500 includes determining one or more binary permutation groups to enable individual differentiation and identification of the wires 101. In various embodiments, the binary permutation groups are determined based at least in part on the total quantity of wires 101 and a sum of consecutive natural numbers. For example, the binary permutation groups may be determined based at least in part on the total quantity of wires 101 and a sum of natural numbers that is equal to or greater than the total quantity of the plurality of wires. The wires 101 may be positioned into binomial permutation groups that are differentiable from one another and enable identification of individual wires 101 based at least in part on the binomial encoding schema. For example, one of the plurality of wires 101 in the helical arrangement 102 may be determined by interpreting the number of the target wire 101 in accordance with the binomial encoding schema while observing the sensor guide wire.

In various embodiments, the binary permutation groups are determined based at least in part on a second sequence of natural numbers obtained by removing, from the consecutive sequence of natural numbers, a natural number that is equal to a difference between the sum of the consecutive sequence of natural numbers and the total quantity of the wires 101. In various embodiments, the binary permutation groups are further determined based at least in part on the second sequence of natural numbers and the schemas 300, 400 shown in FIGS. 4 and 5, respectively. In some embodiments, the binary permutation groups include respective wires 101 from the second subset or the first subset and the second subset. In this manner, the different identification features of the respective wires 101 in the binary permutation groups may correspond to binomial representations of the natural numbers in the second sequence of natural numbers.

At block 509, the process 500 includes positioning the wires 101 into the helical arrangement 102 around the core wire 103 in accordance with the one or more binary permutation groups determined at block 509. As a result, a sensor guide wire 100 may be obtained. In some embodiments, in instances of fabrication, the process 500 may terminate following block 509.

At block 512, the process 500 includes inserting the sensor guide wire 100 to a target site within a body of a subject. In some embodiments, insertion and navigation of the sensor guide wire 100 is performed under guidance from one or more imaging systems, such as X-ray, magnetic resonance imaging (MRI), computed tomography (CT), and/or the like.

At block 515, the process 500 includes obtaining one or more electrophysiological signals at the target site. For example, a first end of one of the plurality of wires 101 may be in contact with tissue at the target site. The first end may receive an electrophysiological signal from the tissue and conduct the electrophysiological signal along a length of the wire 101 to a second end of the wire 101 (referred to as the “conducting wire”). Multiple wires 101 may obtain the same or different electrophysiological signals independently.

At block 518, the process 500 includes identifying the conducting wire (e.g., the one of the plurality of wires 101 that is conducting electrophysiological signals) in accordance with the one or more binary permutation groups determined at block 509. For example, the conducting wire may be identified based at least in part on the binary encoding schema and the arrangement of the first and second subsets of wires 101 in the helical arrangement 102. In some embodiments, a viewing tool (e.g., a hypotube 106, and/or the like) is slid over the sensor guide wire 100 and aligned over a binary permutation group comprising the conducting wire, where an ablation window of the viewing tool may be directly aligned with the conducting wire.

At block 521, the process 500 includes measuring the electrophysiological signal that is being conducted by the identified wire 101. For example, a measurement device may be electrically connected to the identified wire 101 to receive and record the electrophysiological signal. In some embodiments, the process 500 includes ablating the identified wire 101 to enable an electrical connection to the wire 101. In various embodiments, blocks 512-521 may be performed as a separate process from operations associated with blocks 503-509. For example, functionality of blocks 503-509 may be carried out to fabricate a sensor guide wire 100, and functionality of blocks 512-521 may be carried out to use the sensor guide wire 100 to measure electrophysiological signals within a target site of a subject's body.

The depicted blocks indicate operations of each process. Such operations may be performed in any of a number of ways, including, without limitation, in the order and manner as depicted and described herein. In some embodiments, one or more blocks of any of the processes described herein occur in-between one or more blocks of another process, before one or more blocks of another process, in parallel with one or more blocks of another process, and/or as a sub-process of a second process. Additionally, or alternatively, any of the processes in various embodiments include some or all operational steps described and/or depicted, including one or more optional blocks in some embodiments. With regard to the flowcharts illustrated herein, one or more of the depicted block(s) in some embodiments is/are optional in some, or all, embodiments of the disclosure. It should be appreciated that one or more of the operations of each flowchart may be combinable, replaceable, and/or otherwise altered as described herein.

In some embodiments, the helical arrangement comprises: a first segment based on a first binary permutation group, the first segment comprising a first quantity of wires from the second subset and no wires from the first subset, the first quantity of wires from the second subset being adjacent one another; a second segment based on a second binary permutation group, the second segment comprising a first quantity of wires from the first subset and a second quantity of wires from the second subset, the second quantity of wires from the second subset being adjacent one another; and a third segment based on a third binary permutation group, the third segment comprising a second quantity of wires from the first subset and a third quantity of wires from the second subset; the third quantity of wires from the second subset is less than the second quantity of wires from the second subset; the second quantity of wires from the second subset is less than the first quantity of wires from the second subset; and the second quantity of wires from the first subset is greater than the first quantity of wires from the first subset.

In some embodiments, the first identification feature comprises a first color; and the second identification feature comprises a second color that is different from the first color. In some embodiments, the first identification feature comprises a first color pattern; and the second identification feature comprises a second color pattern that is different from the first color pattern. In some embodiments, the first identification feature comprises a first surface texture; and the second identification feature comprises a second surface texture that is different from the first surface texture. In some embodiments, in the helical arrangement: the first quantity of wires from the second subset are adjacent to one another; the second quantity of wires from the second subset are adjacent to one another; and the third quantity of wires from the second subset are adjacent to one another.

In some embodiments, the plurality of wires comprises at least 12 wires. In some embodiments, a respective wire of the plurality of wires comprises a diameter of about 33.0 to 50.0 microns. In some embodiments, the core wire comprises a diameter of about 50.0 to 100.0 microns. In some embodiments, the helical arrangement comprises a diameter of about 250.0 microns. In some embodiments, a respective wire of the plurality of wires comprises a first end and a second end opposite the first end; the first end comprises a sensor configured to measure electrophysiological signal; and the respective wire is configured to conduct the electrophysiological signal to the second end.

In various embodiments, provided herein are methods for fabricating and using sensor guide wires. An example method for fabricating a sensor guide wire may comprise obtaining the core wire; obtaining the plurality of wires; and positioning the first subset of wires and the second subset of wires into the helical arrangement around the core wire in accordance with the at least one binary permutation group. In some embodiments, the method further comprises determining the at least one binary permutation group based at least in part on: a total quantity of the plurality of wires and a sum of natural numbers that is equal to or greater than the total quantity of the plurality of wires, wherein: a respective binary permutation group represents at least a subset of the natural numbers.

An example method for measuring an electrophysiological signal using the sensor guide wire may comprise inserting the sensor guide wire to a target site within a body of a subject; obtaining an electrophysiological signal of the subject at the target site via a sensor comprised by the respective first end of one of the plurality of wires; identifying the respective second end of the one of the plurality of wires based at least in part on the at least one binary permutation group and at least one of the first identification feature or the first identification feature and the second identification feature; and measuring the electrophysiological signal via the respective second end of the identified one of the plurality of wires.

### CONCLUSION

While some embodiments described herein relate to sensor guide wires, one of ordinary skill in the art will appreciate that the teachings herein may also apply to a wide range of medical procedures and apparatuses. The embodiments described herein may also be scalable to accommodate at least the aforementioned applications. Various components of embodiments described herein can be added, removed, reorganized, modified, duplicated, or the like as one skilled in the art would find convenient and/or necessary to implement a particular application in conjunction with the teachings of the present disclosure. In some embodiments, specialized features, characteristics, materials, components, and/or equipment may be applied in conjunction with the teachings of the present disclosure as one skilled in the art would find convenient and/or necessary to implement a particular application.

Moreover, many modifications and other embodiments of the present disclosure set forth herein will come to mind to one skilled in the art to which this disclosure pertains having the benefit of the teachings presented in the foregoing descriptions and the associated drawings. Therefore, it is to be understood that the present disclosure is not to be limited to the specific embodiments disclosed and that modifications and other embodiments are intended to be included within the scope of any appended claims. Moreover, although the foregoing descriptions and the associated drawings describe example embodiments in the context of certain example combinations of elements and/or functions, it should be appreciated that different combinations of elements and/or functions can be provided by alternative embodiments without departing from the scope of any appended claims. In this regard, for example, different combinations of elements and/or functions than those explicitly described above are also contemplated as can be set forth in some of any appended claims. Although specific terms are employed herein, they are used in a generic and descriptive sense only and not for purposes of limitation.

1. A sensor guide wire, comprising:
a core wire extending between a first end and a second end, the first end and the second end defining a length of the sensor guide wire; and
a plurality of wires in a helical arrangement around the core wire, wherein:
a first subset of the plurality of wires comprises an outer surface comprising a first identification feature;
a second subset of the plurality of wires comprises an outer surface comprising a second identification feature that is different from the first identification feature;
respective wires of the first subset and respective wires of the second subset are positioned in the helical arrangement in accordance with at least one binary permutation group of the first identification feature or the first identification feature and the second identification feature to differentiate individual ones of the plurality of wires from one another; and
a quantity of the at least one binary permutation group is based on a total quantity of the plurality of wires.

2. The sensor guide wire of claim 1, wherein:
the helical arrangement comprises:
a first segment based on a first binary permutation group, the first segment comprising a first quantity of wires from the second subset and no wires from the first subset, the first quantity of wires from the second subset being adjacent one another;
a second segment based on a second binary permutation group, the second segment comprising a first quantity of wires from the first subset and a second quantity of wires from the second subset, the second quantity of wires from the second subset being adjacent one another; and
a third segment based on a third binary permutation group, the third segment comprising a second quantity of wires from the first subset and a third quantity of wires from the second subset;
the third quantity of wires from the second subset is less than the second quantity of wires from the second subset;
the second quantity of wires from the second subset is less than the first quantity of wires from the second subset; and
the second quantity of wires from the first subset is greater than the first quantity of wires from the first subset.

3. The sensor guide wire of claim 1, wherein:
the first identification feature comprises a first color; and
the second identification feature comprises a second color that is different from the first color.

4. The sensor guide wire of claim 1, wherein:
the first identification feature comprises a first color pattern; and
the second identification feature comprises a second color pattern that is different from the first color pattern.

5. The sensor guide wire of claim 1, wherein:
the first identification feature comprises a first surface texture; and
the second identification feature comprises a second surface texture that is different from the first surface texture.

6. The sensor guide wire of claim 2, wherein, in the helical arrangement:
the first quantity of wires from the second subset are adjacent to one another;
the second quantity of wires from the second subset are adjacent to one another; and
the third quantity of wires from the second subset are adjacent to one another.

7. The sensor guide wire of claim 2, wherein:
the plurality of wires comprises at least 12 wires.

8. The sensor guide wire of claim 1, wherein:
a respective wire of the plurality of wires comprises a diameter of about 33.0 to 50.0 microns.

9. The sensor guide wire of claim 1, wherein:
the core wire comprises a diameter of about 50.0 to 100.0 microns.

10. The sensor guide wire of claim 1, wherein:
the helical arrangement comprises a diameter of about 250.0 microns.

11. The sensor guide wire of claim 1, wherein:
a respective wire of the plurality of wires comprises a first end and a second end opposite the first end;
the first end comprises a sensor configured to measure electrophysiological signal; and
the respective wire is configured to conduct the electrophysiological signal to the second end.

12. A method for fabricating the sensor guide wire of claim 1, comprising:
obtaining the core wire;
obtaining the plurality of wires; and
positioning the first subset of wires and the second subset of wires into the helical arrangement around the core wire in accordance with the at least one binary permutation group.

13. The method of claim 12, further comprising:
determining the at least one binary permutation group based at least in part on:
a total quantity of the plurality of wires and a sum of natural numbers that is equal to or greater than the total quantity of the plurality of wires, wherein:
a respective binary permutation group represents at least a subset of the natural numbers.

14. A method for measuring an electrophysiological signal using the sensor guide wire of claim 11, comprising:
inserting the sensor guide wire to a target site within a body of a subject;
obtaining an electrophysiological signal of the subject at the target site via a sensor comprised by the respective first end of one of the plurality of wires;
identifying the respective second end of the one of the plurality of wires based at least in part on the at least one binary permutation group and at least one of the first identification feature or the first identification feature and the second identification feature; and
measuring the electrophysiological signal via the respective second end of the identified one of the plurality of wires.
